(19) United States
(12) Patent Application Publication
(10) Pub. No.: US 2023/0233430 A1
(43) Pub. Date: Jul. 27, 2023

(54) POLYOL ESTERS FROM BIOBASED FEEDSTOCKS FOR USE AS EMOLLIENT IN PERSONAL CARE APPLICATIONS

(71) Applicant: PETROLIAM NASIONAL BERHAD (PETRONAS), Kuala Lumpur (MY)

(72) Inventors: Normawati SAMSODIN, Kuala Lumpur (MY); Sara SHAHRUDDIN, Kuala Lumpur (MY); Navin SHARMA KARAM CHAND, Kuala Lumpur (MY); Noor Fadhila Syahida GHAZALI, Kuala Lumpur (MY); Nur Amalina SAMSUDIN, Kuala Lumpur (MY); Nazri MUSLIM, Kuala Lumpur (MY); Adaham ISHAK M, Kuala Lumpur (MY)

(21) Appl. No.: 18/007,586

(22) PCT Filed: Jun. 4, 2021
(86) PCT No.: PCT/MY2021/050044

§ 371 Date: Dec. 1, 2022

(30) Foreign Application Priority Data
Jun. 5, 2020 (MY) PI2020002906

(51) Int. Cl. A61K 8/37; C07C 69/30; C07C 67/08; A61Q 19/00

(52) U.S. Cl. CPC A61K 8/375; C07C 69/30; C07C 67/08; A61Q 19/00; A61K 2800/10

(57) ABSTRACT

Provided are personal care compositions comprising ester polyol esters, ester polyol esters, manufacturing methods thereof, and the use of ester polyol esters in personal care compositions.

### FIELD OF INVENTION

The present invention relates to personal care compositions comprising ester polyol esters, to ester polyol ester compounds, to uses of ester polyol esters in personal care compositions, and to methods of manufacturing ester polyol esters.

### BACKGROUND

The listing or discussion of a prior-published document in this specification should not necessarily be taken as an acknowledgement that the document is part of the state of the art or is common general knowledge.

A common problem associated with personal care compositions such as emollients is an undesirable residue persisting on a person's skin after application of an emollient, particularly an oily or greasy residue. Such residues can cause skin irritation and are unpleasant to a user.

Ester polyol esters are products formed from the reaction of a polyol with more than one equivalents of an ester such that more than one hydroxyl moiety in the polyol is esterified, as described in U.S. Pat. No. 9,885,006 B2. Ester polyol esters are commonly used as lubricants and have not previously been described as useful emollients.

### SUMMARY OF INVENTION

The present inventors have surprisingly found that ester polyol esters can have the necessary properties to be effective emollients. For example, ester polyol esters can coat a person's skin with a thin oil film that keeps skin hydrated but which does not leave an oily residue. Ester polyol esters are also non-irritant, stable and can be produced from renewable feedstocks.

Thus, the present invention provides the following.

1. A personal care composition comprising an ester polyol ester of formula Ia, and an ester polyol ester of formula Ib

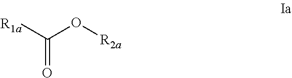

wherein in formula Ia:
each R₁ₐ is independently a linear or branched alkyl chain having from 1 to 17 carbon atoms;
each R₂ₐ is independently a linear or branched alkyl chain having from 2 to 12 carbon atoms substituted by one or more R_{1a′}C(O)O— groups;

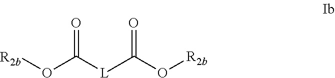

wherein in formula Ib:
L is a linear alkylene chain with from 1 to 18 carbon atoms;
each R_{2b} is independently a linear or branched alkyl chain having from 2 to 12 carbon atoms substituted by one or more R_{1a″}C(O)O— groups; and
each R_{1a′} and R_{1a″} are independently a linear or branched alkyl chain having from 1 to 17 carbon atoms.

2. The personal care composition according to Clause 1, wherein the ester polyol ester of formula Ia is an ester polyol ester of formula Ic:

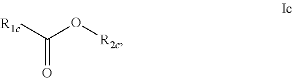

wherein
each R_{1c} is independently the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids, and
each R_{2c} is independently a moiety formed by removing a primary hydroxyl group from a primary polyol selected from the group consisting of: glycerin, diglycerin, ethylene glycol, diethylene glycol, 1,2-propanediol, bis(1,2-propanediol), 2-methyl-1,3-propanediol (2-MePG), trimethylolpropane (TMP), di-trimethylolpropane (Di-TMP), neopentyl glycol (NPG), pentaerythritol (PE), dipentaerythritol (diPE) and sorbitol, wherein said moiety is bonded to the rest of the molecule at the location of the removed primary hydroxyl group, and each remaining hydroxyl functional group in said moiety is esterified with a monocarboxylic acid of formula R_{1c}—COOH.

3. The personal care composition according to Clause 2, wherein each R_{1c} is independently the alkyl chain of a monocarboxylic acid selected from the group consisting of: hexanoic, nonanoic, 2-ethyl hexanoic, heptanoic, octanoic, decanoic, and lauric acids, optionally wherein each R_{1c} is independently the alkyl chain of a monocarboxylic acid selected from the group consisting of octanoic acid and decanoic acid.

4. The personal care composition according to Clause 3, wherein R_{1c} is the alkyl chain of octanoic acid.

5. A personal care composition according to any one of the preceding clauses, wherein ester polyol ester of formula Ib is an ester polyol ester of formula Id:

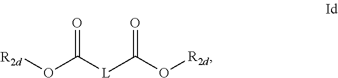

wherein:
L is a linear alkylene chain with from 2 to 14 carbon atoms;
each R_{2d} is independently a moiety formed by removing a primary hydroxyl group from a primary polyol selected from the group consisting of: glycerin, diglycerin, ethylene glycol, diethylene glycol, 1,2-propanediol, bis(1,2-propanediol), 2-methyl-1,3-propanediol (2-MePG), trimethylolpropane (TMP), di-trimethylolpropane (Di-TMP), neopentyl glycol (NPG), pentaerythritol (PE), dipentaerythritol (diPE) and sorbitol, wherein said moiety is bonded to the rest of the molecule at the location of the removed primary hydroxyl group, and each remaining hydroxyl functional group in said moiety is esterified with a monocarboxylic acid of formula R_{1c}—COOH;
each R_{1c} is independently the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids, optionally wherein each R_{1c} is independently the alkyl chain of a monocarboxylic acid selected from the group consisting of octanoic acid and decanoic acid.

6. The personal care composition according to Clause 5, wherein L is a linear alkylene chain with from 4 to 12 carbon atoms.

7. The personal care composition according to Clause 5, wherein L is a C₈ linear alkylene chain.

8. The personal care composition according to any one of the Clauses 2 to 7, wherein R_{2c} and/or R_{2d} are a moiety having the formula:

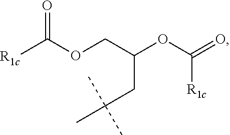

where the dashed line represents the point of attachment to the rest of the molecule, and wherein each R_{1c} independently represents the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids, optionally wherein each R_{1c} is independently the alkyl chain of a monocarboxylic acid selected from the group consisting of octanoic acid and decanoic acid.

9. The personal care composition according to any one of the Clauses 2 to 7, wherein R_{2c} and/or R_{2d} are a moiety having the formula:

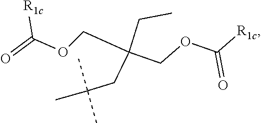

where the dashed line represents the point of attachment to the rest of the molecule, and wherein each R_{1c} independently represents the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids, optionally wherein each R_{1c} is independently the alkyl chain of a monocarboxylic acid selected from the group consisting of octanoic acid and decanoic acid.

10. The personal care composition according to Clause 1, wherein the ester polyol ester of formula Ia is an ester polyol ester of formula IIa, and/or the ester polyol ester of formula Ib is an ester polyol ester of formula IIb:

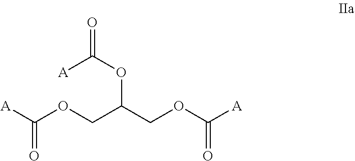

wherein in formula IIa each A independently represents a linear C₅₋₉ alkyl chain, or a linear or branched C₁₄₋₁₇ alkyl chain;

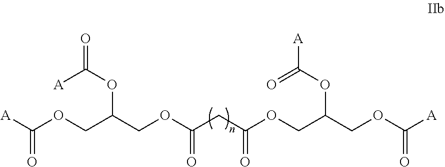

wherein in formula IIb:

each A independently represents a linear C₅₋₉ alkyl chain, or a linear or branched C₁₄₋₁₇ alkyl chain; and

n is an integer of from 6 to 10.

11. The personal care composition according to Clause 10, wherein in formula IIa and/or IIb:

(i) each A independently represents a C₆₋₈ alkyl group, optionally a C₇ alkyl group; or

(ii) each A independently represents the alkyl chain of a monocarboxylic acid selected from the group consisting of palmitic and stearic acids, optionally the alkyl chain of isostearic or stearic acid, more optionally the alkyl chain of isostearic acid.

12. The personal care composition according to Clause 10 or 11, wherein n is an integer of from 7 to 9, optionally wherein n is 8.

13. The personal care composition according to any one of Clauses 10 to 12, wherein each A represents a C₇ alkyl group and n is 8.

14. The personal care composition according to Clause 1, wherein the ester polyol ester of formula Ia is an ester polyol ester of formula IIc, and/or the ester polyol ester of formula Ib is an ester polyol ester of formula IId:

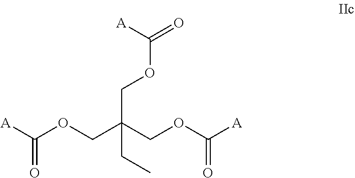

wherein in formula IIc each A independently represents a linear C₅₋₉ alkyl chain, or a linear or branched C₁₄₋₁₇ alkyl chain;

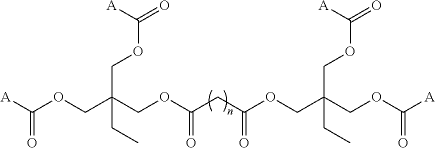

IId

wherein in formula IId:

each A independently represents a linear C₅₋₉ alkyl chain, or a linear or branched C₁₄₋₁₇ alkyl chain; and

n is an integer of from 6 to 10.

15. The personal care composition according to Clause 13, wherein in formula IIc and/or IId:

(i) each A independently represents a C₆₋₈ alkyl group, optionally a C₇ alkyl group; or

(ii) each A independently represents the alkyl chain of a monocarboxylic acid selected from the group consisting of palmitic and stearic acids, optionally the alkyl chain of isostearic or stearic acid, more optionally the alkyl chain of isostearic acid.

16. The personal care composition according to Clause 13 or 14, wherein n is an integer of from 7 to 9, optionally wherein n is 8.

17. The personal care composition according to any one of Clauses 13 to 15, wherein:

(i) each A represents a C₇ alkyl group and n is 8; or

(ii) the compound of formula IIc is trimethylolpropane triisostearate.

18. The personal care composition according to any one of the preceding clauses, wherein the weight ratio of ester polyol ester of formula Ia, Ic, IIa or IIc to ester polyol ester of formula Ib, Id, IIb or IId is from 95:5 to 30:70.

19. The personal care composition according to Clause 18, wherein:

(a) the weight ratio of ester polyol ester of formula Ia, Ic or IIa to ester polyol ester of formula Ib, Id or IIb is from 90:10 to 80:20;

(b) the weight ratio of ester polyol ester of formula Ia, Ic or IIa to ester polyol ester of formula Ib, Id or IIb is from 60:40 to 70:30; or

(c) the weight ratio of ester polyol ester of formula Ia, Ic or IIa to ester polyol ester of formula Ib, Id or IIb is from 40:60 to 50:50.

20. The personal care composition according to any one of the preceding clauses, wherein the ester polyol esters of formula Ia, Ib, Ic, Id, IIa, IIb, IIc or IId are present in the personal care composition in the ratio produced by a reaction between:
a polyol of formula R₂ₐ—OH or R_{2b}—OH;
a monocarboxylic acid of formula R₁ₐ—COOH; and
a dicarboxylic acid of formula HOOC-L-COOH,

wherein the molar ratio of monocarboxylic acid to dicarboxylic acid in the reaction mixture is from 99.99:0.01 to 99:1 (such as about 99.97:0.03 to about 99.7:0.3, for example about 99.95:0.05, about 99.875:0.125, or about 99.78:0.22), and

the ratio of hydroxyl groups to carboxyl groups in the reaction mixture is from about 0.940 to about 0.955.

21. The personal care composition according to Clause 20, wherein the reaction is quenched once the acid value reaches a value below 20 mgKOH/g, and the hydroxyl value reaches a value below 5 mgKOH/g.

22. The personal care composition according to Clause 20 or 21, wherein the mixture of ester polyol esters of formula Ia, Ib, Ic, Id, IIa, IIb, IIc or IId produced by said reaction has a dynamic viscosity at 25° C. of from 20 to 90 cps, preferably a dynamic viscosity at 25° C. selected from:

(a) 20-30 cps;

(b) 35-45 cps; or

(c) 80-90 cps.

23. The personal care composition according to any one of the preceding clauses, wherein the total combined mass percent of ester polyol ester or formula Ia, Ic, IIa or IIc and ester polyol ester of formula Ib, Id, IIb or IId in the personal care composition is from 0.5 to 65 wt. %, preferably from 0.5 to 15 wt. %, such as 0.5 to 12 wt. %.

24. The personal care composition according to any one of Clauses 1, 2, 5, 8 and 9, wherein the ester polyol esters of formula Ia, Ib, Ic, Id, IIa or IIb are derived from a bio-based feedstock, optionally wherein the bio-based feedstock comprises palm acids.

25. The personal care composition according to any one of the preceding clauses, which is an emollient composition.

26. Use of an ester polyol ester as described in any one of Clauses 1 to 25 in a personal care composition, optionally wherein the ester polyol ester comprises a first ester polyol ester or formula Ia, Ic, IIa or IIc and second ester polyol ester of formula Ib, Id, IIb or IId.

27. A compound of formula IIa or 11c:

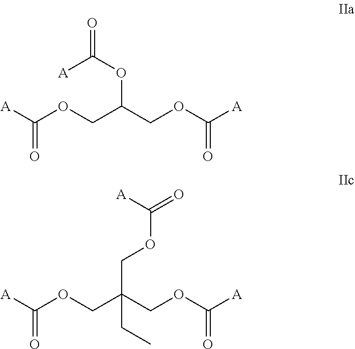

wherein each A independently represents a C₆₋₈ alkyl group.

28. A compound according to Clause 27, which has the structure:

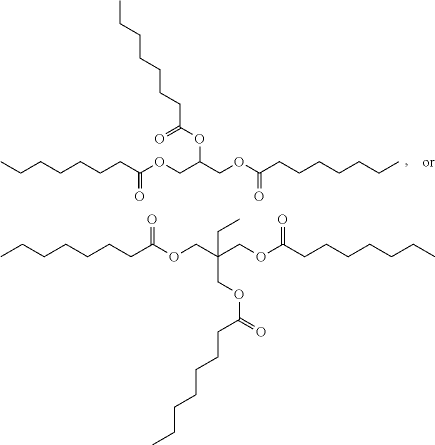

29. A compound of formula IIb or IId:

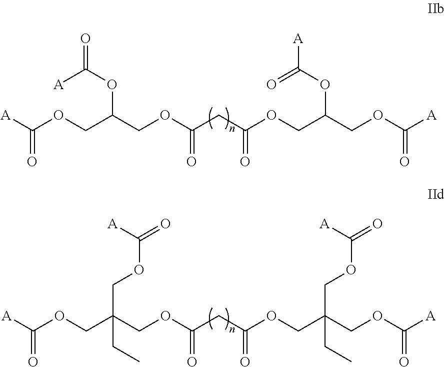

wherein each A independently represents a C₆₋₈ alkyl group and n is an integer of from 6 to 10.

30. A compound according to Clause 29, which has the structure:

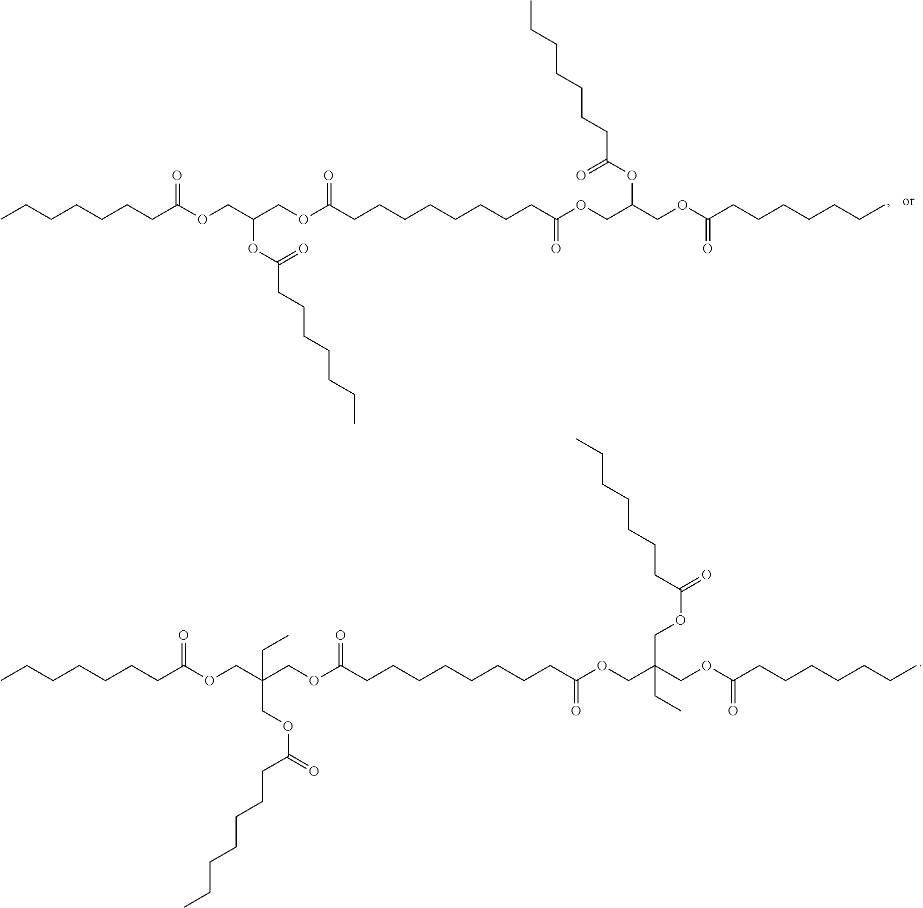

31. A method for preparing an ester polyol ester, the method comprising preparing a reaction mixture comprising:
a polyol selected from the group consisting of glycerin, trimethylolpropane and a combination thereof;
sebacic acid; and
octanoic acid,

wherein the method comprises a step of esterifying the glycerin with sebacic acid and octanoic acid,
wherein the reaction mixture has a hydroxyl group to carboxyl group ratio (HCR) corresponding to a ratio of moles of hydroxyl groups to moles of carboxyl groups, and the HCR is less than about 1, and wherein the molar ratio of octanoic acid to sebacic acid is from 99.99:0.01 to 99:1 (such as about 99.97:0.03 to about 99.7:0.3, for example about 99.95:0.05, about 99.875:0.125, or about 99.78:0.22).

32. The method according to Clause 31, wherein the reaction mixture has a hydroxyl to carboxyl ratio (HCR) of less than about 0.97, preferably from about 0.92 to about 1, more preferably from about 0.92 to about 0.97, further preferably from about 0.93 to about 0.95, more preferably still from about 0.940 to about 0.955.

33. The method according to Clause 31 or 32, wherein the reaction is quenched once the acid value reaches a value below 20 mgKOH/g, and the hydroxyl value reaches a value below 5 mgKOH/g.

34. The method according to any one of Clauses 31 to 33, wherein the mixture of ester polyol esters produced by said method has a dynamic viscosity at 25° C. of from 20 to 90 cps, preferably a dynamic viscosity at 25° C. selected from:

(a) 20-30 cps;

(b) 35-45 cps; or

(c) 80-90 cps.

### DRAWINGS

FIG. 1 shows the structure of an ester polyol ester formed from glycerin and caprylic acid.

FIG. 2 shows the structure of an ester polyol ester formed from glycerin, sebacic acid and caprylic acid.

FIG. 3 shows toxicity data for compounds according to the invention as tested with OECD 492.

FIG. 4 shows toxicity data for compounds according to the invention as tested with OECD 439. Certain embodiments of the present disclosure are described more fully hereinafter with reference to the accompanying drawings.

### DESCRIPTION

The invention provides a personal care composition comprising an ester polyol ester of formula Ia, and an ester polyol ester of formula Ib

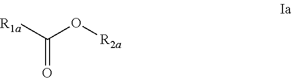

wherein in formula Ia:
each Ria is independently a linear or branched alkyl chain having from 1 to 17 carbon atoms;
each R₂ₐ is independently a linear or branched alkyl chain having from 2 to 12 carbon atoms substituted by one or more R₁ₐ C(O)O— groups;

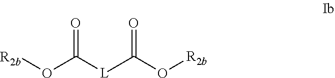

wherein in formula Ib:
L is a linear alkylene chain with from 1 to 18 carbon atoms;
each R_{2b} is independently a linear or branched alkyl chain having from 2 to 12 carbon atoms substituted by one or more R₁ₐ C(O)O— groups; and
each R_{1a′} and R_{1a″} are independently a linear or branched alkyl chain having from 1 to 17 carbon atoms.

In embodiments herein, the term “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

In embodiments herein, the term “ester polyol ester” means a compound formed from the esterification of at least two hydroxyl groups in a polyol. In embodiments of the invention, the ester polyol esters have at least 90% esterification of polyol hydroxyl groups, such as at least 95%, at least 99%, at least 99.5% such as about 100% esterification of polyol hydroxyl groups. The term “polyol ester” may be used interchangeably herein with “ester polyol ester”.

In embodiments herein, the term “alkyl” means a monovalent group of formula —CₙH₂ₙ₊₁. Alkyl groups may be linear or branched. In some embodiments of the invention, alkyl groups may be linear. In other embodiments of the invention, alkyl groups may be branched.

In embodiments herein, the term “alkylene” means a divalent group of formula —CₙH₂ₙ—. Alkylene groups may be linear or branched. In some embodiments of the invention, alkylene groups are typically linear.

The terms “formulation” and “composition” may be used interchangeably herein.

In some embodiments of the invention, the ester polyol ester of formula Ia may be an ester polyol ester of formula Ic:

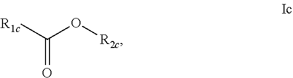

wherein each R_{1c} is independently the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids, and each R_{2c} is independently a moiety formed by removing a primary hydroxyl group from a primary polyol selected from the group consisting of: glycerin, diglycerin, ethylene glycol, diethylene glycol, 1,2-propanediol, bis(1,2-propanediol), 2-methyl-1,3-propanediol (2-MePG), trimethylolpropane (TMP), di-trimethylolpropane (Di-TMP), neopentyl glycol (NPG), pentaerythritol (PE), dipentaerythritol (diPE) and sorbitol, wherein said moiety is bonded to the rest of the molecule at the location of the removed primary hydroxyl group, and each remaining hydroxyl functional group in said moiety is esterified with a monocarboxylic acid of formula R_{1c}—COOH.

In some aspects of these embodiments, each R_{1c} may independently be the alkyl chain of a monocarboxylic acid selected from the group consisting of: hexanoic, nonanoic, 2-ethyl hexanoic, heptanoic, octanoic, decanoic, and lauric acids. In particular aspects of these embodiments, each R_{1c} may independently be the alkyl chain of a monocarboxylic acid selected from the group consisting of octanoic acid and decanoic acid. For example, R_{1c} may be the alkyl chain of octanoic acid.

In some embodiments of the invention, the ester polyol ester of formula Ib may be an ester polyol ester of formula Id:

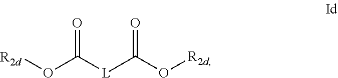

wherein L is a linear alkylene chain with from 2 to 14 carbon atoms;

each R_{2d} is independently a moiety formed by removing a primary hydroxyl group from a primary polyol selected from the group consisting of: glycerin, diglycerin, ethylene glycol, diethylene glycol, 1,2-propanediol, bis(1,2-propanediol), 2-methyl-1,3-propanediol (2-MePG), trimethylolpropane (TMP), di-trimethylolpropane (Di-TMP), neopentyl glycol (NPG), pentaerythritol (PE), dipentaerythritol (diPE) and sorbitol, wherein said moiety is bonded to the rest of the molecule at the location of the removed primary hydroxyl group, and each remaining hydroxyl functional group in said moiety is esterified with a monocarboxylic acid of formula R_{1c}—COOH;

each R_{1c} is independently the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids. In some aspects of these embodiments, each R_{1c} may independently be the alkyl chain of a monocarboxylic acid selected from the group consisting of octanoic acid and decanoic acid.

In some aspects of these embodiments, L may be a linear alkylene chain with from 4 to 12 carbon atoms, for example a C₈ linear alkylene chain.

In some aspects of the above embodiments of the invention, R_{2c} and/or R_{2d} may be a moiety having the formula:

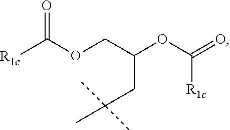

where the dashed line represents the point of attachment to the rest of the molecule, and wherein each R_{1c} independently represents the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids. In some aspects of these embodiments, each R_{1c} may independently be the alkyl chain of a monocarboxylic acid selected from the group consisting of octanoic acid and decanoic acid.

In some other aspects of the above embodiments of the invention, R_{2c} and/or R_{2d} may be a moiety having the formula:

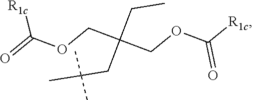

where the dashed line represents the point of attachment to the rest of the molecule, and wherein each R_{1c} independently represents the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids. In some aspects of these embodiments, each R_{1c} may independently be the alkyl chain of a monocarboxylic acid selected from the group consisting of octanoic acid and decanoic acid.

In some embodiments of the invention, the ester polyol ester of formula Ia may be an ester polyol ester of formula IIa, and/or the ester polyol ester of formula Ib is an ester polyol ester of formula IIb:

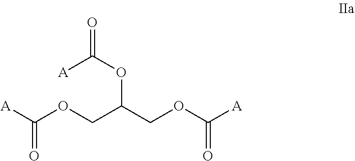

wherein in formula IIa each A independently represents a linear C₅₋₉ alkyl chain, or a linear or branched C₁₄₋₁₇ alkyl chain;

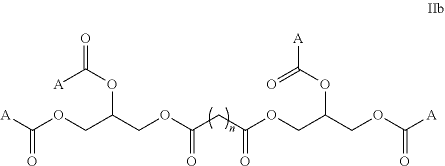

wherein in formula IIb:

each A independently represents a linear C₅₋₉ alkyl chain, or a linear or branched C₁₄₋₁₇ alkyl chain; and

n is an integer of from 6 to 10.

In some aspects of these embodiments, each A may independently represent a linear C₅₋₉ alkyl chain, for example a linear C₆₋₈ alkyl group, such as a linear C₇ alkyl group. In some aspects of these embodiments, n is an integer of from 7 to 9, such as 8. In a particular aspect of these embodiments, each A represents a C₇ alkyl group and n is 8. A particular example of a compound of formula IIb that may be mentioned herein is capryloyl glycerin/sebacic acid copolymer.

In other aspects, each A may independently represent a linear or branched C₁₄₋₁₇ alkyl chain, optionally the alkyl chain of a monocarboxylic acid selected from the group consisting of palmitic and stearic acids, more optionally the alkyl chain of isostearic or stearic acid, further optionally the alkyl chain of isostearic acid. In these aspects, n is an integer of from 6 to 10, optionally an integer of from 7 to 9, such as 8.

In some embodiments of the invention the ester polyol ester of formula Ia may be an ester polyol ester of formula IIc, and/or the ester polyol ester of formula Ib may be an ester polyol ester of formula IId:

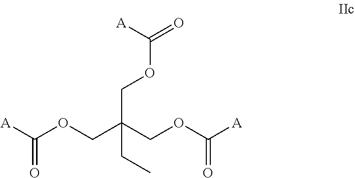

wherein in formula IIc each A independently represents a linear C₅₋₉ alkyl chain, or a linear or branched C₁₄₋₁₇ alkyl chain;

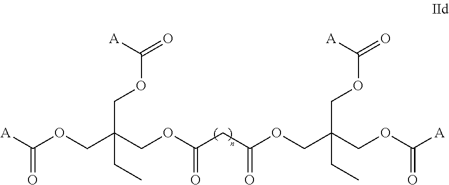

wherein in formula IId:

each A independently represents a linear C₅₋₉ alkyl chain, or a linear or branched C₁₄₋₁₇ alkyl chain; and

n is an integer of from 6 to 10.

In some aspects of these embodiments, each A may independently represent a linear C₅₋₉ alkyl chain, for example a linear C₆₋₈ alkyl group, such as a linear C₇ alkyl group. In some aspects of these embodiments, n is an integer of from 7 to 9, such as 8. In a particular aspect of these embodiments, each A represents a C₇ alkyl group and n is 8.

In other aspects, each A may independently represent a linear or branched C₁₄₋₁₇ alkyl chain, optionally the alkyl chain of a monocarboxylic acid selected from the group consisting of palmitic and stearic acids, more optionally the alkyl chain of isostearic or stearic acid, further optionally the alkyl chain of isostearic acid. In these aspects, n is an integer of from 6 to 10, optionally an integer of from 7 to 9, such as 8.

A particular example of a compound of formula IIc that may be mentioned herein is trimethylolpropane triisostearate.

The weight ratio of the ester polyol ester of formula Ia, Ic, IIa or IIc to ester polyol ester of formula Ib, Id, IIb or IId may be from 95:5 to 30:70. The particular weight ratio will influence the properties of the mixture of ester polyol esters, such as the viscosity and surface tension. The ratio of ester polyol esters may be varied to provide the desired physical properties for the personal care composition, which will depend on the type of personal care composition. For example, as will be appreciated by a person skilled in the art, the required physical properties for a hair wax are different to those for a moisturising cream.

Particular weight ratios of ester polyol esters that may be useful in the present invention are set out below.
(a) The weight ratio of ester polyol ester of formula Ia, Ic or IIa to ester polyol ester of formula Ib, Id or IIb is from 90:10 to 80:20.
(b) The weight ratio of ester polyol ester of formula Ia, Ic or IIa to ester polyol ester of formula Ib, Id or IIb is from 60:40 to 70:30.
(c) The weight ratio of ester polyol ester of formula Ia, Ic or IIa to ester polyol ester of formula Ib, Id or IIb is from 40:60 to 50:50.

Typically, the ratio of ester polyol esters in a personal care composition will correspond to the ratio in which they are produced during the production method. The ratio of ester polyol ester of formula Ia, Ic, IIa or IIc to ester polyol ester of formula Ib, Id, IIb or IId produced by the reaction will depend on the ratio of monocarboxylic acid (e.g. octanoic acid) to dicarboxylic acid (e.g. sebacic acid) in the reaction mixture. The greater the amount of dicarboxylic acid, the greater the proportion of ester polyol ester of formula Ib, Id, IIb or IId. Since the presence of dicarboxylic acid can result in the formation of oligomers (and if present in sufficiently high amounts, polymers) which are undesirable in an emollient, the concentration of dicarboxylic acid is typically kept low. For example, the molar ratio of monocarboxylic acid to dicarboxylic acid in the reaction mixture is typically from 99.99:0.01 to 99:1 (such as about 99.97:0.03 to about 99.7:0.3). Specific molar ratios that may be mentioned herein include about 99.95:0.05, about 99.875:0.125, and about 99.78:0.22.

Therefore, in some embodiments of the invention, the the ester polyol esters of formula Ia, Ib, Ic, Id, IIa, IIb, IIc or IId are present in the personal care composition in the ratio produced by a reaction between:
a polyol of formula R₂ₐ—OH or R_{2b}—OH;
a monocarboxylic acid of formula R₁ₐ—COOH; and
a dicarboxylic acid of formula HOOC-L-COOH,

wherein the molar ratio of monocarboxylic acid to dicarboxylic acid in the reaction mixture is from 99.99:0.01 to 99:1 (such as about 99.97:0.03 to about 99.7:0.3, for example about 99.95:0.05, about 99.875:0.125, or about 99.78:0.22), and

the ratio of hydroxyl groups to carboxyl groups in the reaction mixture is from about 0.940 to about 0.955.

In some embodiments of the invention, the reaction to produce the ester polyol esters is quenched when the acid value reaches a value below 20 mgKOH/g, and the hydroxyl value reaches a value below 5 mgKOH/g. This has the effect of stopping the reaction when the amount of free acid and hydroxyl groups is low, and the reaction has almost run to completion. This may help to reduce the formation of unwanted side products.

The mixture of ester polyol esters produced by the reaction will have a dynamic viscosity. This is a key property that influences how the personal care composition will feel on a person's skin, and can be adjusted by varying the carbon chain lengths and ratio of mono-acid to diacid in the reaction mixture. In some embodiments of the invention, ester polyol esters of formula Ia, Ib, Ic, Id, IIa, IIb, IIc or IId produced by said reaction has a dynamic viscosity at 25° C. of from 20 to 90 cps. Particular dynamic viscosities at 25° C. that may be mentioned herein include 20-30 cps; 35-45 cps; and 80-90 cps.

The amount of ester polyol ester in a personal care composition will depend on the intended use of the personal care composition. In his context, “amount of ester polyol ester” refers to the total amount of ester polyol ester of formula Ia, Ib, Ic, Id, IIa, IIb, IIc and IId in the composition. Typically, the amount of ester polyol ester in the personal care composition may be from 0.5 to 65 wt. %. In some embodiments of the invention that may be mentioned herein, the amount of ester polyol ester in the personal care composition may be from about 0.5 to about 15 wt. %, such as 0.5 to 12 wt. %. Particular personal care compositions according to the invention are discussed in the below Examples.

As mentioned herein, an advantage of the invention is that the ester polyol esters may be produced from a variety of feedstocks. For example, they may be produced from renewable biobased feedstocks. They may also be produced from synthetic or non-biobased feedstocks. In some embodiments of the invention, particularly when longer chain acids are desired, the ester polyol esters may be formed from a biobased feedstock comprising palm acids, which may be derived from palm oil.

The personal care composition of the invention may be any personal care composition in which the ester polyol esters may be useful. For example, the personal care composition may be a haircare composition or a skincare composition, especially an emollient composition. Examples of haircare compositions include a hair mask, a hair wax, a shampoo, and a hair conditioner. Examples of a skincare composition include a body wash, a skin or body lotion, and a body milk. Any of the personal care compositions of the invention may be optimised for healthy adult skin, baby skin, or other sensitive skin. In particular, a body wash, skin or body lotion, shampoo and body milk may be desirably optimised for baby skin or other sensitive skin such as on a user's face. A person skilled in the art will appreciate that some personal care compositions may be suitable for more than one use, and may function as both a haircare and skincare composition, or have dual functionality within the class of haircare or skincare compositions. Examples of dual functionality personal care compositions include a combined hair and body wash, and a combined “two in one” shampoo and conditioner composition.

For the avoidance of doubt, when any numerical range is used herein, the higher and lower values of any related ranges may be combined to provide new ranges, which are all specifically contemplated herein. For example, the ester polyol esters used in the personal care compositions may have a dynamic viscosity at 25° C. selected from any one of the below ranges:
from 20 to 90 cps, from 20 to 80 cps, from 20 to 45 cps, from 20 to 35 cps, from 20 to 30 cps;
from 30 to 90 cps, from 30 to 80 cps, from 30 to 45 cps, from 30 to 35 cps;
from 35 to 90 cps, from 35 to 80 cps, from 35 to 45 cps;
from 45 to 90 cps, from 45 to 80 cps;
from 80 to 90 cps.

The invention also provides the use of an ester polyol ester as disclosed herein, in a personal care composition. The use may comprise the use of a first ester polyol ester or formula Ia, Ic, IIa or IIc and second ester polyol ester of formula Ib, Id, IIb or IId.

The invention also provides certain compounds of formula IIa, IIb, IIc and IId. In particular the invention provides a compound of formula IIa or IIc:

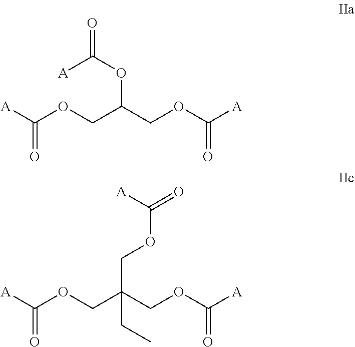

wherein each A independently represents a C₆₋₈ alkyl group.

In some embodiments of the invention, the compound of formula IIa may have the formula

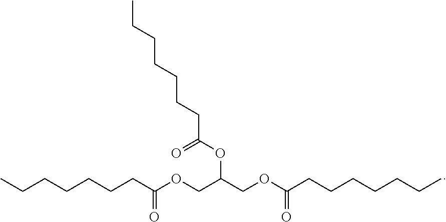 [SMILES: CCCCCCCC(=O)OCC(COC(=O)CCCCCCC)OC(=O)CCCCCCC]

In some embodiments of the invention, the compound of formula IIc may have the formula

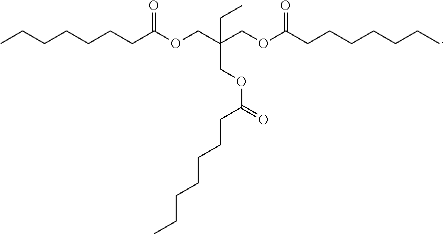 [SMILES: CCCCCCCC(=O)OCC(CC)(COC(=O)CCCCCCC)COC(=O)CCCCCCC]

The invention provides a compound of formula IIb or IId:

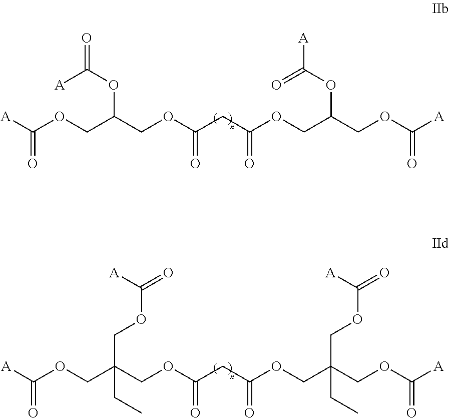

wherein each A independently represents a C₆₋₈ alkyl group and n is an integer of from 6 to 10.

In some embodiments of the invention, the compound of formula IIb has the structure

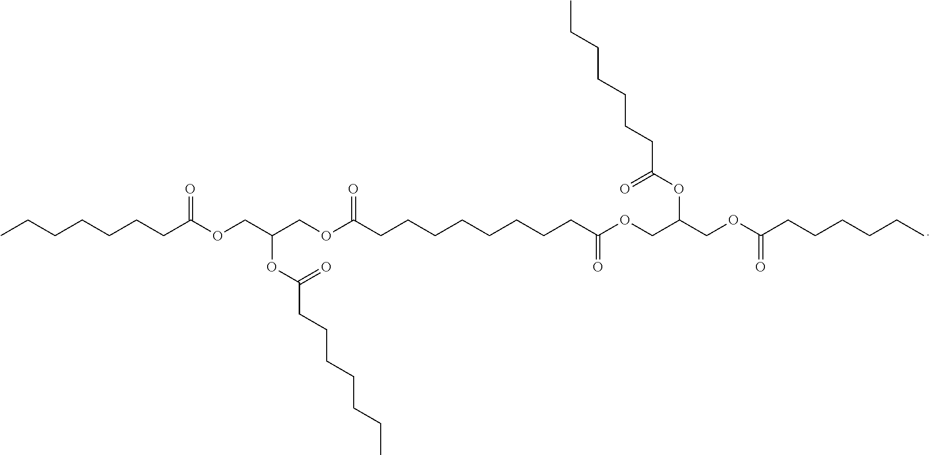 [SMILES: CCCCCCCC(=O)OCC(COC(=O)CCCCCCCCC(=O)OCC(COC(=O)CCCCCC)OC(=O)CCCCCCC)OC(=O)CCCCCCC]

In some embodiments of the invention, the compound of formula IId has the structure

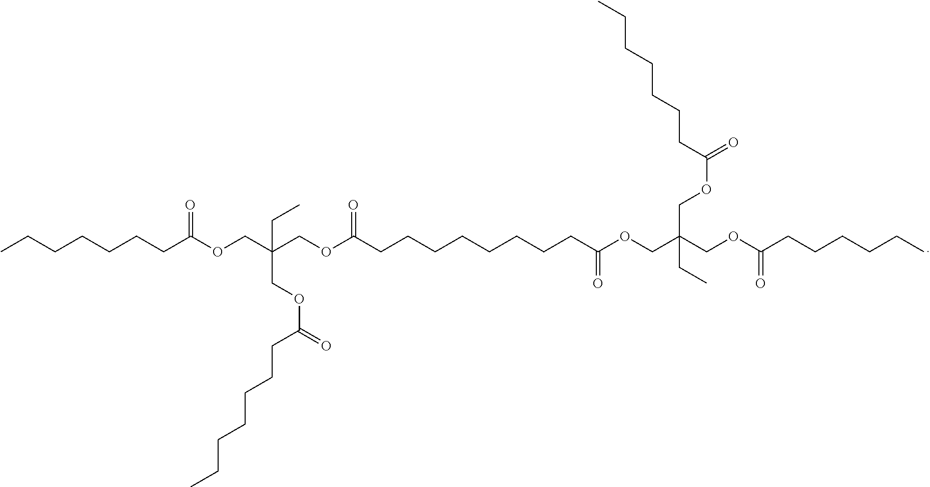 [SMILES: CCCCCCCC(=O)OCC(CC)(COC(=O)CCCCCC)COC(=O)CCCCCCCCC(=O)OCC(CC)(COC(=O)CCCCCCC)COC(=O)CCCCCCC]

The invention also provides a method for preparing an ester polyol ester (such as an ester polyol ester of formula IIa-IId above), the method comprising preparing a reaction mixture comprising:
a polyol selected from the group consisting of glycerin, trimetholoyl propane and a combination thereof;
sebacic acid; and
octanoic acid,

wherein the method comprises a step of esterifying the glycerin with sebacic acid and octanoic acid,
wherein the reaction mixture has a hydroxyl group to carboxyl group ratio (HCR) corresponding to a ratio of moles of hydroxyl groups to moles of carboxyl groups, and the HCR is less than about 1, and wherein the molar ratio of octanoic acid to sebacic acid is from 99.99:0.01 to 99:1.

The molar ratio of octanoic acid to sebacic acid may be about 99.97:0.03 to about 99.7:0.3, for example about 99.95:0.05, about 99.875:0.125, or about 99.78:0.22). The reaction may be quenched once the acid value reaches a value below 20 mgKOH/g, and the hydroxyl value reaches a value below 5 mgKOH/g. Typically, the mixture of ester polyol esters produced by the reaction may have a dynamic viscosity at 25° C. of from 20 to 90 cps. For example, the mixture of ester polyol esters produced by the reaction may have a dynamic viscosity at 25° C. of from 20-30 cps, 35-45 cps, or 80-90 cps.

As will be appreciated by a person skilled in the art, other ester polyol esters useful in the personal care compositions of the invention may be made by corresponding methods to that described above. Other methods for preparing ester polyol esters are described in U.S. Pat. No. 9,885,006 B2 and the below Examples.

The ester polyol esters used in the present invention can provide personal care compositions having enhanced sensory characteristics. For example, the ester polyol esters are more fluid than many known emollients, and this provides a lighter and less oily fee on the skin, as well as resulting in lower irritation potential. The ester polyol esters are also highly stable, enhancing the oxidation stability of the personal care compositions and making them suitable for prolonged storage without requiring antioxidants to be added to the composition.

Ester polyol esters formed from polyols such as glycerin and TMP are highly branched, allowing the manufacture of higher molecular weight products that have lower viscosity than unbranched ester polyol esters. This improves the spreadability of the resulting compositions, providing the properties discussed above. The ester polyol esters may also have a higher refractive index, resulting in a higher shine, which is especially desirable in haircare products.

The below Examples illustrate the invention, and are not to be construed as limitative.

### EXAMPLES

Materials and Methods

Sebacic acid was purchased from Megachem, with purity 99.5%. Caprylic acid was purchased from KLK Oleochemicals with purity 99%. Glycerin was purchased from KLK Oleochemicals with purity 99.7%.

Acid value was determined as per ASTM D664.

### Example 1: Reaction Procedure for the Synthesis of Polyol Esters

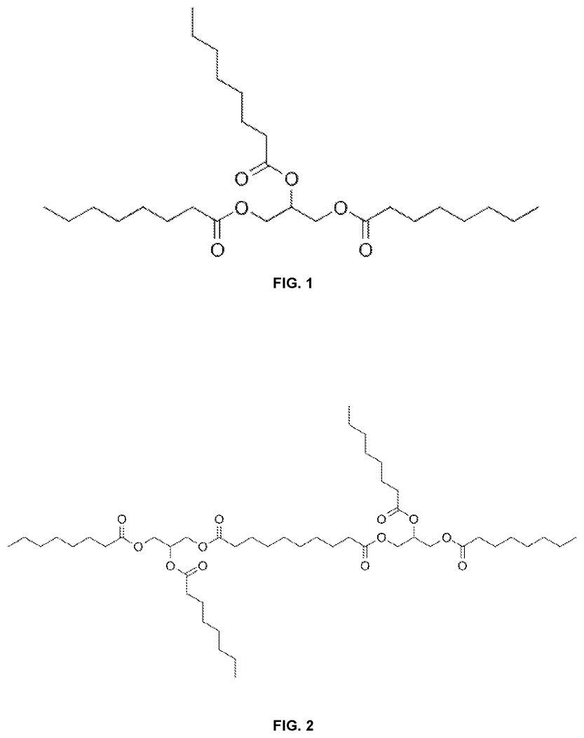

A polyol, a monoacid, a diacid, and a tin oxide catalyst (amounts specified in Tables 1a and 1 b below) were mixed in a reactor protected from oxygen and moisture. The reaction setup comprised a 4-neck round bottom flask equipped with overhead stirrer, inlet for nitrogen gas for blanketing, condenser with dean stark apparatus attached to it and a thermocouple/temperature controller. The resulting mixture was initially homogenized at 115° C., after which the content was sampled to determine the acid value. The reactor temperature was increased stepwise to 115° C., 180° C. and finally to the maximum reaction temperature of 210° C. The reaction produced water, which was removed by the elevated temperature and condensed by a condenser. When the rate of water production from condensation was slowed and the overhead temperature was reduced, the reactor was sparged with nitrogen at a rate of 0.2 L/min to accelerate removal of water. The reaction was progressed until the acid value reached 20 mgKOH/g was reached. Unreacted fatty acid and catalyst was removed by alkaline and clay treatment as described in U.S. Pat. No. 9,885,006 B2. Briefly, unreacted acid was removed by the minimum amount of the weakest strength alkaline material necessary to achieve the desired acid value. Residual catalyst was removed using Montmorillonite clay adsorbent having a high surface area resulting in excellent adsorption properties. The as-synthesized mixture of ester polyol esters was assigned the references identified in Tables 1a and 1b. PE25, PE50 and PE100 comprise the two compounds shown in FIG. 1 and FIG. 2. EM25 and EM44 comprise analogous compounds formed from the C₉ mono- and di-acids azelaic acid and pelargonic acid.

The amount of each reagent used to prepare the glycerin ester polyol esters is shown in Table 1a.

TABLE 1a
Details of reactants for preparing glycerin polyol esters

| | PE25 | | PE50 | | PE100 | |
|---|---|---|---|---|---|---|
| Glycerin (g) | 60.03 | [0.6518] | 61.83 | [0.6714] | 63.67 | [0.6914] |
| [moles] | | | | | | |
| Sebacic acid (g) | 19.00 | [0.0939] | 42.98 | [0.2125] | 67.51 | [0.4681] |
| [moles] | | | | | | |
| Caprylic acid (g) | 270.97 | [1.8790] | 245.19 | [1.7002] | 218.81 | [1.5173] |
| [moles] | | | | | | |

| | | | |
|---|---|---|---|
| Tin oxide catalyst | 450 | 450 | 450 |
| (ppm) | | | |
| Diacid to | 0.05 | 0.125 | 0.22 |
| monoacid weight | | | |
| ratio | | | |
| Hydroxyl to | 0.9461 | 0.9477 | 0.9493 |
| carboxyl ratio | | | |

Corresponding esters made from trimethylolpropane were produced by equivalent methods, using the starting materials listed in Table 1 b below.

TABLE 1b
Details of reactants for preparing TMP polyol esters

| | EM25 | EM44 |
|---|---|---|
| TMP (moles) | 0.15972 | 0.16770 |
| Azelaic Acid | 0.01429 | 0.05122 |
| (moles) | | |
| Pelargonic Acid | 0.47949 | 0.42887 |
| (moles) | | |
| Tin oxide catalyst | 300 | 300 |
| (ppm) | | |
| Diacid to | 0.029 | 0.12 |
| monoacid weight | | |
| ratio | | |
| Hydroxyl to | 0.94 | 0.95 |
| carboxyl ratio | | |

### Example 2: Physical and Chemical Properties of the Ester Polyol Esters

Basic physical and chemical analyses were performed on the polyol esters using the identified standard and in-house methods as listed in Table 2. The results of the analysis are tabulated in Table 3 and subsequent sections.

TABLE 2
Tests conducted on polyol esters.

| Description | Method | Purpose |
|---|---|---|
| Appearance | Visual | To observe the appearance of the |
| | | synthesized polyol esters, typically should |
| | | be Bright & Clear (B&C). |
| Acid Value | ASTM D664 | To determine the acid value for reaction |
| | | completion and final product properties. |
| Pour Point | ASTM D97 | To determine the pour point of the |
| | | synthesized polyols esters. |
| Moisture Content | ASTM D6304/ | To determine the water/moisture content of |
| | ASTM D4672 | the synthesized polyol esters |
| Dynamic Viscosity | In-House using | To determine the viscosity of the |
| | Rheometer | synthesized polyol esters. |
| Flash point | ASTM D92 | To determine the flash point of the polyol |
| | | esters. |
| Cloud Point | ASTM D2500 | To determine the cloud point of the polyol |
| | | esters whereby the cloud point is the |
| | | temperature at which wax crystals begin to |
| | | appear in a liquid |

TABLE 3
Physical and chemical properties of the polyol esters

| | | Emollient | | | |
|---|---|---|---|---|---|
| | | specification in | Emollient | | |
| Properties | Unit | personal care | PE25 | PE50 | PE100 |
| Dynamic Viscosity at 25° C. | cps | As reported | 24.46 | 40.63 | 83.99 |
| Acid value | mgKOH/g | <1.0 | 0.01227 | 0.425 | 0.61021 |
| Colour (ASTM D1500) | | <0.5 | <0.5 | <0.5 | <0.5 |
| Lead | | <20 | 1.86 | <.0.9 | 0.935 |
| Cadmium | | <5 | — | <0.4 | <0.4 |
| Others | ppm | <10 | <10 | <10 | <10 |
| (P, Cr, Cu, Fe, Mn, Mg, | | | | | |
| Sn, V, Zn, Ti, Mo, Ni, | | | | | |
| Na, B, S, Ba, Al, K, Si | | | | | |
| Pour point | ° C. | <−30 | −38 | −49 | −53 |
| Cloud point | ° C. | As reported | −36 | −50 | −44 |
| Spreadability | mm 2/10 | As reported | 573.97 | 440.61 | 286.67 |
| | min | | | | |
| Surface tension | mN/m | <30 | 25.38 | 29.51 | 27.36 |
| pH | | As reportedt | 5 | 5 | 5 |
| Flash Point | ° C. | >150° C. | >150 | >150 | >150 |
| Density | g/cm³ | As reported | 0.81667 | 0.90766 | 0.98098 |

Surface Tension

The elastic-like force between the as-synthesized polyol esters and air, also referred to as surface tension, was determined using the Du Nouy Ring test method. This test is based on the principle that the force required to pull a ring through a liquid is related to the liquid's surface tension. Briefly, a ring attached to a torsion meter was dipped into the as-synthesized polyol ester. The ring was then subsequently withdrawn from the polyol ester, and the force on the ring was measured shortly before a liquid film tears. The surface tension was then calculated from the diameter of the ring and the tear-off force. The results are recorded in Table 4.

Surface tension has a strong impact on the wetting properties of a material and is also correlated with the spreadability of the material together. Typically, emollient with a low surface tension exhibits fast spreading behaviour and vice versa. This is shown by PE25, which has a low measured surface tension and a fast spreading ability, as compared to PE50 and PE100.

TABLE 4
Surface tension of polyol esters

| Sample | Surface Tension (mN/m) |
|---|---|
| PE 25 | 25.38 |
| PE 50 | 29.51 |
| PE 100 | 27.36 |

TGA

The decomposition temperature of the reactants and polyol ester of different grades (PE25, PE50 and PE100) were determined using a thermogravimetric analyser (TGA). TGA is a method of thermal analysis by measuring the changes in the mass of a sample over increasing temperatures.

For reactants caprylic acid and glycerin, more than 99 wt. % of the sample was decomposed within the temperature range as shown in Table 5. However, for sebacic acid, only 95.24 wt. % of the sample was decomposed and its decomposition temperature is higher than the other feedstock being tested. This was attributed to the physical state of sebacic acid which exists as a solid, while the other reactants are in liquid form. It was noted that the boiling points of all reactants are higher than their decomposition temperatures.

TABLE 5
Decomposition temperature range of reactants

| | Decomposition temperature | Boiling point temperature |
|---|---|---|
| | (° C.) | (° C.) |
| Caprylic acid | 150.9 to 193.3 | 237.0 |
| Sebacic acid | 247.9 to 283.9 | 294.4 |
| Glycerin | 178.7 to 220.9 | 290.0 |

The analysis was also carried out on the polyol ester samples PE25, PE50 and PE100. There are two decomposition temperature ranges, indicating the existence of two different oligomeric structures in each sample. The decomposition temperature range for both samples are almost similar, suggesting that the oligomers in both samples have the same molecular structure. Typically, the higher the decomposition temperature, the higher the molecular weight of the oligomer. The results in Table 6 show that PE100 contains a higher percentage of oligomer with higher molecular weight (54.80 wt. %) as compared to PE25 and PE50.

TABLE 6
Decomposition temperature range of polyol esters

| | PE25 | PE 50 | PE 100 |
|---|---|---|---|
| | First decomposition | | |
| Temperature range | 300-327 | 270-333 | 277-356 |
| (° C.) | | | |
| Percentage | 86.82 | 64.32 | 45.36 |
| decomposed (wt. %) | | | |
| | Second decomposition | | |
| Temperature range | 408-497 | 372-447 | 390-427 |
| (° C.) | | | |
| Percentage | 13.55 | 34.32 | 54.80 |
| decomposed (wt. %) | | | |

Spreadability

The spreading ability of the polyol ester samples was measured by the following method. At 25° C., a drop of the sample (20 μl) was applied to a flat, horizontal surface of ash-less paper (a Whatman filter paper). The liquid emollient sample was left to spread for 10 minutes. After 10 minutes, the wetted area was measured and the results are tabulated in Table 7.

PE25 recorded the highest spreading area over a 10 minute duration, followed by PE50 and PE100. PE25 exhibited a fast spreading ability compared to PE50 and PE100 with medium and low spreading ability, respectively. Visually, PE25 appeared to be less viscous than PE50 and PE100.

TABLE 7
Spreadability of polyol esters

| Sample | Spreadability (mm²/10 minutes) |
|---|---|
| PE 25 | 573.97 |
| PE 50 | 440.61 |
| PE 100 | 286.67 |

Inductively Coupled Plasma-Optical Emission Spectrometry (ICP-OES) Analysis

The metal content in the polyol esters was determined using ICP-OES (Perkin Elmer Optima DV 5300). The preparation of apparatus, standards and samples follow the standard operating procedure APHA 3120B. The results are shown in Table 3.

The effectiveness of clay treatment during purification step is verified since the tin content, which is used as catalyst during esterification reaction, is less than 1 mg/kg for all three samples.

TABLE 8
ICP-OES results of the polyol esters

| | Metal content (mg/kg) | | |
|---|---|---|---|
| Element | PE 25 | PE 50 | PE 100 |
| P | 3.98 | 0.839 | 0.633 |
| Cr | — | 0.487 | 0.514 |
| Cu | 1.41 | 0.478 | 0.455 |
| Cd | — | <0.4 | <0.4 |
| Fe | — | 0.672 | 0.477 |
| Mn | 0.04 | 0.448 | 0.174 |
| Mg | 1.88 | <10 | <10 |
| Sn | 0.25 | 0.938 | 0.851 |
| V | 0.27 | 0.76 | 0.605 |
| Zn | — | 1.43 | 1.13 |
| Ti | 0.21 | 0.824 | 0.668 |
| Mo | — | 0.596 | 0.432 |
| Ni | 1.07 | 0.781 | 0.735 |
| Na | 9.06 | <30 | <30 |
| B | 1.60 | 6.00 | 4.02 |
| S | — | 2.59 | 3.88 |
| Ba | — | 1.53 | 0.714 |
| Al | 4.68 | 1.71 | 1.49 |
| K | 16.48 | <7 | <7 |
| Pb | 1.86 | 0.935 | <0.9 |
| Ca | 1.08 | <20 | <20 |
| Si | 13.64 | 1.37 | 14 |
| Li | — | 1.36 | 0.871 |
| Ag | — | <0.1 | <0.1 |

Toxicology

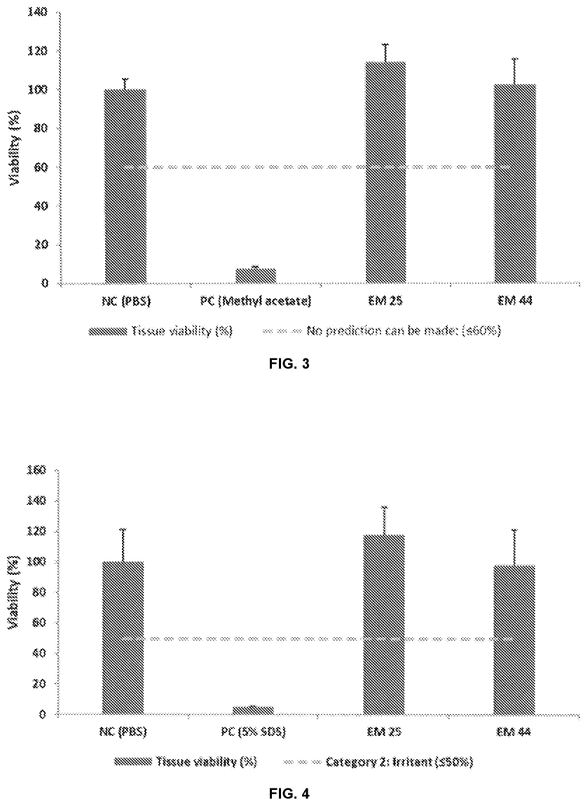

The toxicological properties of the polyol esters EM25 and EM44 were evaluated using the test methods listed below. Results are shown in FIGS. 3 and 4. For OECD 492 a mean tissue viability of greater than or equal to 60% results in no classification, and indicates a non-irritant compound, while a mean tissue viability score of less than 60% results in a classification (Category 1 or Category 2), and indicates an irritant compound. For OECD 439 a mean tissue viability of greater than or equal to 50% results in no classification, and indicates a non-irritant compound, while a mean tissue viability score of less than 50% results in a classification (Category 1 or Category 2), and indicates an irritant compound.
1. In Vitro Eye Irritation: OECD 492
2. Skin Irritation Test: OECD TG439

As shown by the results in FIGS. 3 and 4, both EM25 and EM44 are non-irritant compounds according to both OECD 492 and OECD TG439.

### Example 3: Examples of Personal Care Formulations

Haircare Formulations

TABLE 9
Rinse-off Deep Nutrition Hair Mask formulation
Rinse-off Deep Nutrtion Hair Mask

| Ingredient | Wt % |
|---|---|
| Ester polyol esters | 10.0 |
| Shorea Robusta Seed Butter & Pentaerythrityl Tetra-Di-T- | 10.0 |
| Butyl Hydroxyhydrocinnamate Cetearyl Alcohol & | |
| Behentrimonium Methosulfate | |
| Aqua/Water | Up to 100 |
| Hydrogenated Starch Hydrolysate | 3.0 |
| Glycerin | 3.0 |
| Guar Hydroxypropyltrimonium Chloride | 0.5 |
| Disodium EDTA | 0.1 |
| Chlorphenesin | 0.28 |
| Isopentyldiol | 3.0 |
| Phenoxyethanol | 0.9 |
| Buteth-3 & Sodium Benzotriazolyl Butylphenol Sulfate & | 0.3 |
| Tributyl Citrate | |
| Fragrance | 1.0 |
| Colorant Unicert Yellow 08006 | 2 |
| Colorant Unicert Yellow 08005 | 0.8 |

TABLE 10
Hair Modelling Wax formulation
Hair Modelling Wax

| Ingredient | Wt % |
|---|---|
| Ester polyol esters | 58.10 |
| Hydrogenated Macademia Inegrifolia Seed Oil | 15.0 |
| Cera Alba & Cetyl Palmitate & Stearic Acid | 7.0 |
| Mangifera Indica Seed Butter | 5.0 |
| Behenyl Alcohol | 4.5 |
| Butyrospermum Parkii Butter | 10 |
| Helianthus Anuus Seed Oil & | 0.1 |
| Rosmarinus Officinalis Extract | |
| Fragrance | 0.3 |

TABLE 11
Nature-Derived, Sulfate-Free Moisturizing
Shampoo and Body Wash formulation
Natural-Derived, Sulfate-Free Moisturizing
Shampoo and Body Wash

| Ingredient | Wt % |
|---|---|
| Ester polyol esters | 1.0 |
| D. I. Water | Up to 100 |
| Sodium Lauryl Sulfoacetate and Disodium Laureth | 40.0 |
| Sulfosuccinate | |
| Cocamidopropyl Hydroxysultaine | 6.0 |
| Panthenol | 0.5 |
| Sodium Chloride | q.s. |
| Preservative, Dye, Fragrance & pH adjuster | q.s. |

TABLE 12
Air conditioner formulation
Hair Conditioner

| Ingredient | Wt % |
|---|---|
| Ester polyol esters | 4.0 |
| D. I. Water | Up to 100 |
| Hydroxypropyl Starch Phosphate | 3.0 |
| Quaternium - 82 | 2.0 |
| Cetyl Alcohol | 2.0 |
| Stearyl Alcohol | 2.0 |
| Potassium Chloride | 0.5 |
| Preservative, Dye, Fragrance & pH adjuster | q.s. |

Bodycare Formulations

TABLE 13
Skin Lotion formulation
Skin Lotion

| Ingredient | Wt % |
|---|---|
| Ester polyol esters | 6.0 |
| D. I. Water | Up to 100 |
| Steareth-20 | 40.0 |
| Cetyl Alcohol | 0.5 |
| Glyceryl Stearate | 0.5 |
| Preservative, Dye, Fragrance & pH adjuster | q.s. |

TABLE 14
Sprayable Body Lotion formulation
Sprayable Body Lotion

| Ingredient | Wt % |
|---|---|
| Ester polyol esters | 10 |
| D. I. Water | Up to 100 |
| Glycerin | 1.5 |
| Emulsifying Wax | 1.5 |
| Cetearyl Alcohol | 0.5 |
| Glyceryl Stearate | 1.0 |
| Preservative, Dye, Fragrance & pH adjuster | q.s. |
| Citric Acid | q.s. |
| Sodium Hydroxide | q.s |

Babycare Formulations

TABLE 15
Mild Conditioning Shampoo for Kids formulation
Mild Conditioning Shampoo for Kids

| Ingredient | Wt % |
|---|---|
| Ester polyol esters | 1.5 |
| Aqua/Water | 64.7 |
| Sodium Laureth Sulfate 28% | 8.0 |
| Methoxy PEG/PPG-7/3 Aminopropyl Dimethicone | 0.7 |
| Sodium Cocoamphoacetate | 11.0 |
| Disodium PEG-5 Laurylcitrate Sulfosuccinate, | 10.5 |
| Sodium Laureth Sulfate | |
| Citric Acid (30% in water) | 1.7 |
| Palmitamidopropyltrimonium Chloride | 1.5 |
| Polyquartenium -10 | 0.2 |
| Preservative, Fragrance | q.s. |

TABLE 16
Mild Baby Shampoo formulation
Mild Baby Shampoo

| Ingredient | Wt % |
|---|---|
| Ester polyol esters | 9.5 |
| Aqua/Water | 65.8 |
| Sodium Laureth Sulfate 28% | 15.0 |
| Cocamidopropyl Betaine, Glyceryl Laurate | 10.0 |
| Sodium Chloride | q.s. |
| Preservative, Fragrance | q.s. |

TABLE 17
Moisturizing Light Body Milk formulation
Moisturizing Light Body Milk

| Ingredient | Wt % |
|---|---|
| Ester polyol esters | 4.0 |
| Aqua/Water | 89.0 |
| Isoamyl Cocoate | 3.5 |
| Ceramide NP | 0.1 |
| Carbomer | 0.2 |
| Sodium Hydroxide (10% in water) | q.s. |
| Phenolxyethanol, Ethylhexylglycerin | 0.7 |
| Polyglyceryl-6 Stearate and Polyglyceryl-6 Behenate | 3.0 |
| Preservative, Fragrance | q.s. |

### Example 4: Comparison of Various Chemical Properties with Known Emollients

TABLE 18
Comparison of chemical properties with known emollients.

| | | | 3T | Crodamol TTIS |
|---|---|---|---|---|
| | | | [Commercial product | [Commercial product |
| | | | from Stepan] | from Croda) |
| | | | (Comparative | (Comparative |
| Name | EM25 | EM44 | Example) | Example) |
| Molecular | 849 | 1186 | 546 | 950 |
| Weight | | | | |
| Viscosity | 26 | 49 | 25 | 167 |
| Spreadability | Fast | Medium | Fast | Medium |
| Refractive Index | 1.452 | 1.455 | 1.451 | 1.463 |

The ester polyol esters EM25 and EM44 have improved viscosity, spreadability and refractive index for their molecular weight. This results in improved sensory characteristics and an advantageously increased shine.

1. A personal care composition comprising an ester polyol ester of formula Ia, and an ester polyol ester of formula Ib 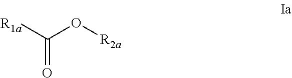
wherein in formula Ia:
each R₁ₐ is independently a linear or branched alkyl chain having from 1 to 17 carbon atoms;
each R₂ₐ is independently a linear or branched alkyl chain having from 2 to 12 carbon atoms substituted by one or more R_{1a′}C(O)O— groups; 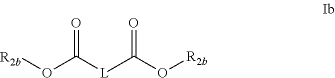
wherein in formula Ib:
L is a linear alkylene chain with from 1 to 18 carbon atoms;
each R_{2b} is independently a linear or branched alkyl chain having from 2 to 12 carbon atoms substituted by one or more R_{1a″}C(O)O— groups; and
each R_{1a′} and R_{1a″} are independently a linear or branched alkyl chain having from 1 to 17 carbon atoms.

2. The personal care composition according to claim 1, wherein the ester polyol ester of formula Ia is an ester polyol ester of formula Ic: 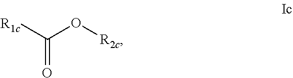
wherein
each R_{1c} is independently the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids, and each R_{2c} is independently a moiety formed by removing a primary hydroxyl group from a primary polyol selected from the group consisting of: glycerin, diglycerin, ethylene glycol, diethylene glycol, 1,2-propanediol, bis(1,2-propanediol), 2-methyl-1,3-propanediol (2-MePG), trimethylolpropane (TMP), di-trimethylolpropane (Di-TMP), neopentyl glycol (NPG), pentaerythritol (PE), dipentaerythritol (diPE) and sorbitol, wherein said moiety is bonded to the rest of the molecule at the location of the removed primary hydroxyl group, and each remaining hydroxyl functional group in said moiety is esterified with a monocarboxylic acid of formula R_{1c}—COOH.

3. The personal care composition according to claim 2, wherein each R_{1c} is independently the alkyl chain of a monocarboxylic acid selected from the group consisting of: hexanoic, nonanoic, 2-ethyl hexanoic, heptanoic, octanoic, decanoic, and lauric acids.

4. (canceled)

5. A personal care composition according to claim 1, wherein ester polyol ester of formula Ib is an ester polyol ester of formula Id: 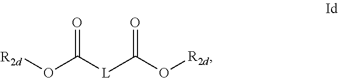
wherein:
L is a linear alkylene chain with from 2 to 14 carbon atoms;
each R_{2d} is independently a moiety formed by removing a primary hydroxyl group from a primary polyol selected from the group consisting of: glycerin, diglycerin, ethylene glycol, diethylene glycol, 1,2-propanediol, bis(1,2-propanediol), 2-methyl-1,3-propanediol (2-MePG), trimethylolpropane (TMP), di-trimethylolpropane (Di-TMP), neopentyl glycol (NPG), pentaerythritol (PE), dipentaerythritol (diPE) and sorbitol, wherein said moiety is bonded to the rest of the molecule at the location of the removed primary hydroxyl group, and each remaining hydroxyl functional group in said moiety is esterified with a monocarboxylic acid of formula R_{1c}—COOH;
each R_{1c} is independently the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids.

6. The personal care composition according to claim 5, wherein L is a linear alkylene chain with from 4 to 12 carbon atoms.

7. (canceled)

8. The personal care composition according to claim 2, wherein R_{2c} is a moiety having the formula: 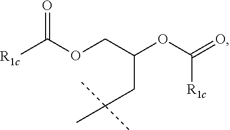
where the dashed line represents the point of attachment to the rest of the molecule, and wherein each R_{1c} independently represents the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids.

9. The personal care composition according to claim 2, wherein R_{2c} is a moiety having the formula: 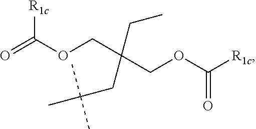
where the dashed line represents the point of attachment to the rest of the molecule, and wherein each R_{1c} independently represents the alkyl chain of a monocarboxylic acid selected from the group consisting of: acetic, propanoic, butyric, pentanoic, hexanoic, nonanoic, 2-ethyl hexanoic, heptanoic, octanoic, decanoic, lauric, myristic, palmitic and stearic acids.

10. The personal care composition according to claim 1, wherein the ester polyol ester of formula Ia is an ester polyol ester of formula IIa, and/or the ester polyol ester of formula Ib is an ester polyol ester of formula IIb: 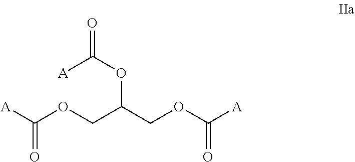
wherein in formula Ha each A independently represents a linear C₅₋₉ alkyl chain, or a linear or branched C₁₄₋₁₇ alkyl chain; 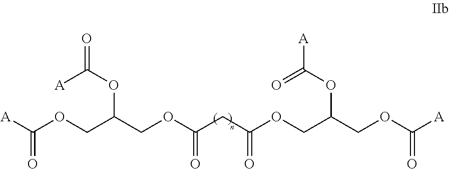
wherein in formula IIb:
each A independently represents a linear C₅₋₉ alkyl chain, or a linear or branched C₁₄₋₁₇ alkyl chain; and
n is an integer of from 6 to 10.

11. The personal care composition according to claim 10, wherein in formula IIa and/or IIb:
(i) each A independently represents a C₆₋₈ alkyl group; or
(ii) each A independently represents the alkyl chain of a monocarboxylic acid selected from the group consisting of palmitic and stearic acids.

12. The personal care composition according to claim 10, wherein n is an integer of from 7 to 9.

13. (canceled)

14. The personal care composition according to claim 1, wherein the ester polyol ester of formula Ia is an ester polyol ester of formula IIc, and/or the ester polyol ester of formula Ib is an ester polyol ester of formula IId: 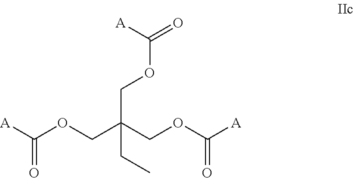
wherein in formula IIa each A independently represents a linear C₅₋₉ alkyl chain, or a linear or branched C₁₄₋₁₇ alkyl chain; 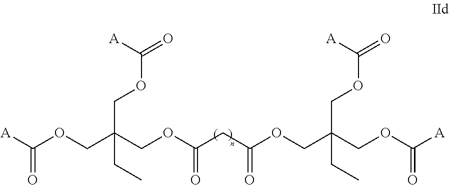
wherein in formula IIb:
each A independently represents a linear C₅₋₉ alkyl chain, or a linear or branched C₁₄₋₁₇ alkyl chain; and
n is an integer of from 6 to 10.

15. The personal care composition according to claim 14, wherein in formula IIc and/or IId:
(i) each A independently represents a C₆₋₈ alkyl group; or
(ii) each A independently represents the alkyl chain of a monocarboxylic acid selected from the group consisting of palmitic and stearic acids.

16. (canceled)

17. The personal care composition according to claim 14, wherein:
(i) each A represents a C₇ alkyl group and n is 8; or
(ii) the compound of formula IIc is trimethylolpropane triisostearate.

18. The personal care composition according to claim 1, wherein the weight ratio of ester polyol ester of formula Ia to ester polyol ester of formula Ib is selected from:
(a) from 95:5 to 30:70;
(b) from 90:10 to 80:20;
(c) from 60:40 to 70:30; or
(d) from 40:60 to 50:50.

19. (canceled)

20. The personal care composition according to claim 1, wherein the ester polyol esters of formula Ia or Ib are present in the personal care composition in the ratio produced by a reaction between:
a polyol of formula R₂ₐ—OH or R_{2b}—OH;
a monocarboxylic acid of formula R₁ₐ—COOH; and
a dicarboxylic acid of formula HOOC-L-COOH,
wherein the molar ratio of monocarboxylic acid to dicarboxylic acid in the reaction mixture is from 99.99:0.01 to 99:1, and
the ratio of hydroxyl groups to carboxyl groups in the reaction mixture is from about 0.940 to about 0.955.

21. (canceled)

22. (canceled)

23. The personal care composition according to claim 1, wherein one or more of the following apply:
(a) the total combined mass percent of ester polyol ester or formula Ia and ester polyol ester of formula Ib in the personal care composition is from 0.5 to 65 wt. %;
(b) the ester polyol esters of formula Ia or Ib are derived from a bio-based feedstock; or
(c) the personal care composition is an emollient composition.

24. (canceled)

25. (canceled)

26. Use of an ester polyol ester as described in claim 1 in a personal care composition.

27. A compound of formula IIa, IIb, IIc or IId: 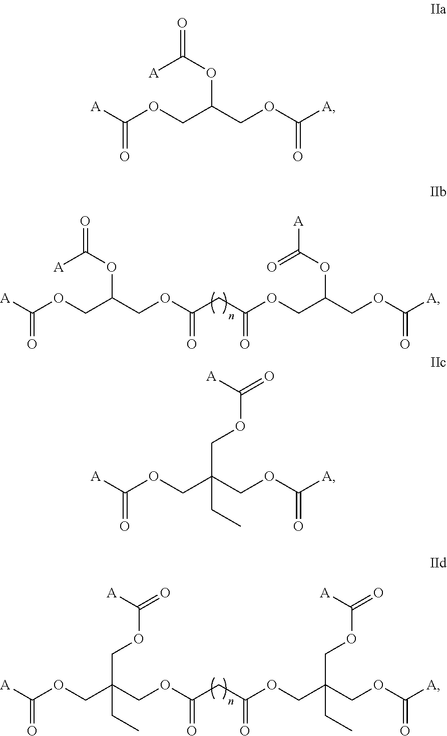
wherein each A independently represents a C₆₋₈ alkyl group and n is an integer of from 6 to 10.

28. A compound according to claim 27, which has the structure:

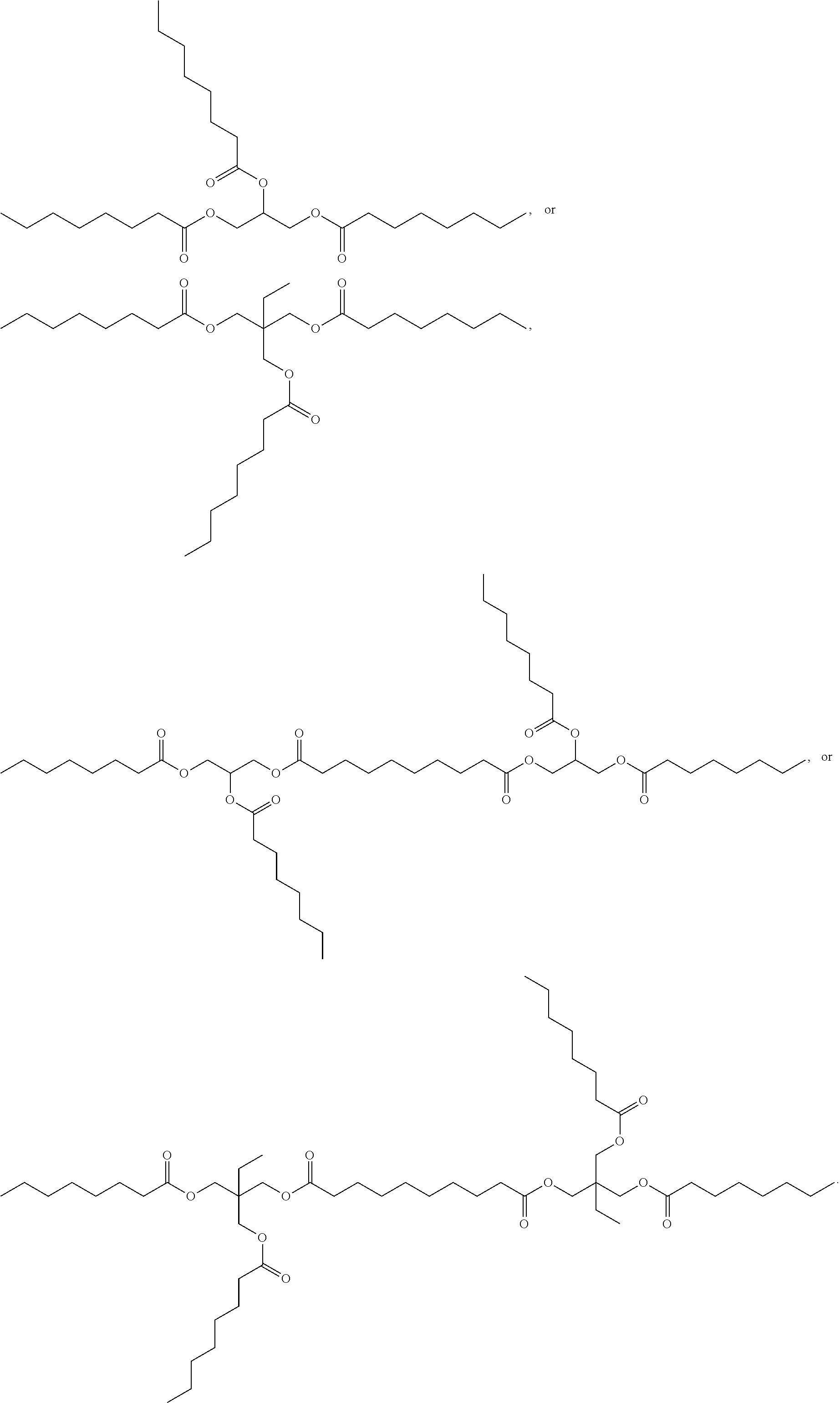

29. (canceled)

30. (canceled)

31. A method for preparing an ester polyol ester, the method comprising preparing a reaction mixture comprising:
a polyol selected from the group consisting of glycerin, trimethylolpropane and a combination thereof;
sebacic acid; and
octanoic acid,
wherein the method comprises a step of esterifying the glycerin with sebacic acid and octanoic acid,
wherein the reaction mixture has a hydroxyl group to carboxyl group ratio (HCR) corresponding to a ratio of moles of hydroxyl groups to moles of carboxyl groups, and the HCR is less than about 1, and wherein the molar ratio of octanoic acid to sebacic acid is from 99.99:0.01 to 99:1.

32-34. (canceled)
